(19) United States
(12) Patent Application Publication
(10) Pub. No.: US 2022/0047880 A1
(43) Pub. Date: Feb. 17, 2022

(54) DEVICE FOR GENERATING COLD PLASMA COMPRISING ELECTRODES AND DIELECTRICS

(71) Applicant: L'OREAL, Paris (FR)

(72) Inventors: Thi Hong Lien PLANARD-LUONG, Chevilly Larue (FR); Matthieu JACOB, Chevilly Larue (FR)

(73) Assignee: L'OREAL, Paris (FR)

(21) Appl. No.: 17/278,870

(22) PCT Filed: Sep. 26, 2018
(86) PCT No.: PCT/EP2018/076167

§ 371 Date: Mar. 23, 2021

(51) Int. Cl. A61N 1/44; H05H 1/24; A61N 1/04

(52) U.S. Cl. CPC A61N 1/44; A61N 1/0472; A61N 1/0408; H05H 1/2406

(57) ABSTRACT

A treatment device for a body surface to be treated using a dielectric barrier plasma, including a high-voltage generator connected to a headpiece having a conductive element at least partially shielded by a dielectric wall that forms at least one part of an end wall facing the surface to be treated.

### FIELD OF THE INVENTION

The invention relates to a treatment device for a body surface to be treated using a dielectric barrier plasma, comprising a headpiece which has an end wall and an electrode shielded from the surface to be treated by a dielectric.

The invention also relates to a process comprising moving the treatment device on the skin as well as to the use of the treatment device for preventing and/or treating aesthetic disorders of the healthy scalp or of the healthy skin.

By “cosmetic product” is meant any product as defined in Regulation (EC) No. 1223/2009 of the European Parliament and of the Council of 30 Nov. 2009 on cosmetic products.

### BACKGROUND OF THE INVENTION

Systems delivering atmospheric cold plasma have been used for some years in the medical field for applications such as sterilization and treatment of infected wounds. These systems are mainly based on the so-called DBD (Dielectric Barrier Discharge) plasma production technique. These DBD systems, applied to air, are known to produce reactive species of oxygen and nitrogen that have effects on living cells, including antimicrobial effects.

Several patents have been filed describing systems to ensure an air gap between the applicator and the skin.

KR101662156 describes a skin treatment apparatus for treating an affected area using plasma by inducing dielectric barrier discharge between the affected area and a plasma generator and thus producing soft plasma. The skin treatment apparatus uses a ball type plasma generator which promotes sterilization of the affected area and growth of granulation tissues by making a portion touching the affected area in a ball shape headpiece. The ball type plasma generator smoothly moves and operates inside and outside cells at the same time and generates reaction species, thereby increasing concentration of the reaction species and stimulating the affected area.

DE 10 2009 060 627 describes an electrode arrangement made of a planar, flexible electrode and a flexible, planar dielectric, in which the dielectric surrounds the planar electrode on all sides and only one connector of the electrode is routed out of the dielectric, in an insulating manner, for connection to a high-voltage generator. The dielectric is intended to be placed on the surface to be treated, for example the skin surface of a human or animal body, and includes a studded structure on the, contact side, which functions as a spacer, because gas chambers can form between the studs, in which the dielectric barrier plasma can be formed.

A treatment device according to DE 10 2012 015 482 is equipped with a similar electrode arrangement, wherein the dielectric embedding the electrode forms the end wall of a housing of a treatment device. The flexible electrode arrangement made of the flexible dielectric including the flexibly embedded, planar electrode is pressed against the surface to be treated by an elastic pressing means situated behind the electrode arrangement, whereby the adaptability of the electrode arrangement to contours of the surface to be treated, in particular the skin surface, is improved.

DE102015111401 describes a treatment device for a surface to be treated using a dielectric barrier plasma, comprising a housing which has an end wall; an electrode which is shielded from the surface to be treated by a dielectric that forms at least one part of the end wall. The electrode is connectable to a high-voltage generator. The end wall has at least one spacer which forms at least one gas chamber when the at least one spacer rests against the surface to be treated. The dielectric barrier plasma is formed in the gas chamber. A storage chamber finable with a treatment agent arranged on a side of the end wall faces away from the surface to be treated.

The problem addressed by the present invention is to provide a treatment device of the type mentioned at the outset in order to provide a more uniform plasma application and generation on a target such as the skin.

### SUMMARY OF THE INVENTION

In order to solve this problem, the invention proposes a treatment device for a body surface to be treated using a dielectric barrier plasma, comprising a high-voltage generator connected to a headpiece comprising a conductive element at least partially shielded by a dielectric wall that forms at least one part of an end wall facing the surface to be treated, characterized by the fact that the conductive element is provided with one or more projecting conductive elements and the dielectric wall conforms to the shape of the projecting conductive elements.

According to the invention, the conductive element can be an electrode or a noble gas ionized by an electrical field. In the case of a noble ionized gas, the device might comprise a head piece forming an ampule or a bleb.

The treatment device according to the invention therefore makes it possible to avoid that the skin rushes into the space between the spacers according to the prior art, which has the effect of plugging the applicator by depriving it of air and thus prevent the generation of plasma.

Thus, regardless of the depression of each projecting conductive element in the skin, there will always be an air gap between the dielectric barrier and the skin, ensuring a sufficient distance between the skin and the high voltage electrode for generating a plasma.

An intermediate space forms between the dielectric shielding the electrode and the surface to be treated. The dielectric barrier plasma forms a gas chamber or air chamber via ionization of the gas or air that is present there. As a matter of course, a direct current flow is generated between the surface to be treated and the electrode impeded by the dielectric. Therefore, only displacement currents are possible for the plasma formation.

The plasma formation can take place using a DC high voltage, only an initial displacement current results and the potential difference maintaining the plasma. The use of an AC high voltage is preferred, however, if the high-voltage potential switches between a positive voltage and a negative voltage. It is preferred, in this case, that the surface to be treated, for example the skin surface or the body, functions as a so-called floating counter electrode. It could only sluggishly follow the change in potential of the AC voltage and, therefore, due to the changing frequency, essentially remains at an average potential which will become the ground potential.

Another object of the invention is a cosmetic process for treating a body surface using a dielectric barrier plasma, a device according to the invention being moved on the body surface. The process of the invention is a dynamic process. A dynamic routine of the applicator then allows a homogenization of the application of plasma on the skin. In addition, the dynamic routine is facilitated by the roundness of the elements of the applicator.

Another object of the invention is the use of a device according to the invention for preventing and/or treating aesthetic disorders of the healthy scalp or of the healthy skin.

### Main Definitions

The “axis A” means the longitudinal axis A.

A “longitudinal section of axis A” is a section parallel to the axis A of the part.

A “cross section of a part of axis A” is a section perpendicular to the axis A of the part.

The expression “longitudinal axis of the part x” denotes the line connecting all of the centers of mass of the cross sections of the part x.

The term “length of a part A” designates the longitudinal direction of a part of axis A, and the term “width” designates the perpendicular direction.

For example, the “length of the electrode in longitudinal cross section” refers to the longitudinal axis of the electrode.

A “relief” comprises a plurality of parts that protrude out of a surface.

The “thickness of the dielectric wall” is measured perpendicularly to the tangent to the external wall of the dielectric wall.

A “plasma” is an ionised gas which can also generate reactive species, produced by the discharge resulting in the plasma. A cold atmospheric plasma is a plasma which does not cause any excessive heating of the substrate exposed to the plasma.

Three families of cold atmospheric plasmas are available, namely direct, indirect and hybrid plasmas.

In direct plasmas, it is the substrate which acts as the counter electrode required for producing the plasma; the plasma is therefore generated between the electrode and the substrate. One of the main direct plasma technologies is the plasma known as DBD (Dielectric Barrier Discharge).

For the purposes of the present invention, the term “preventing” means reducing the risk of manifestation of the phenomenon under consideration.

Insulants or dielectrics are materials with a very high resistivity: 10⁸ to 10¹⁶ am, because they contain few free electrons. According to the invention, the dielectric wall is a solid material.

Examples of dielectric material usable according to the invention are glass, ceramics, mica, papers, fabrics, organic resins.

### PREFERRED EMBODIMENTS

According to further advantageous aspects of the invention, the device comprises one or several of the following features taken in isolation or in any technically possible combinations:
The conductive element is an electrode. This is a first embodiment of the device according to the invention
The conductive element is an ionized noble gas. This is a second embodiment according to the invention. This is a second embodiment of the device according to the invention.
An external surface of the end wall is provided with a plurality of reliefs.
The end wall delimits with the body surface to be treated one or more gas chambers when the end walls bears on the surface to be treated, the dielectric barrier plasma being generated in the gas chambers. The plasma is formed homogenously in the chamber.
The thickness of the dielectric element is constant over the whole surface of the end wall facing the surface to be treated. The treatment is safe.
The reliefs comprise a curved external surface. Such a structure is more comfortable for the user.
The projecting conductive elements have a concave surface with a concavity turned towards the electrode. Such a surface is soft for the skin of the user.
The projecting conductive elements have a circular, elliptical, oval or polygonal cross section perpendicular to their longitudinal axis.
The plasma is of direct type, in particular DBD type.
The plasma is generative violet ray.
The reliefs are distributed within the external surface of the end wall. The relief serve as a bearing surface.
The reliefs are distributed at the periphery of the external surface of the end wall. The volume of the gas chamber is bigger for some special treatments.
The reliefs are regularly distributed on the external surface of the end wall. The result of the treatment is similar all over the treated surface.

### BRIEF DESCRIPTION OF THE DRAWINGS

The invention may be better understood from reading the following detailed description of non-limiting implementation examples thereof, and with reference to the appended drawing, schematic and partial, in which:

FIG. 1 shows a view of a first embodiment of a treatment device according to the invention in the assembled state;

FIG. 2 shows an enlarged representation of the headpiece of FIG. 1 in longitudinal cross section.

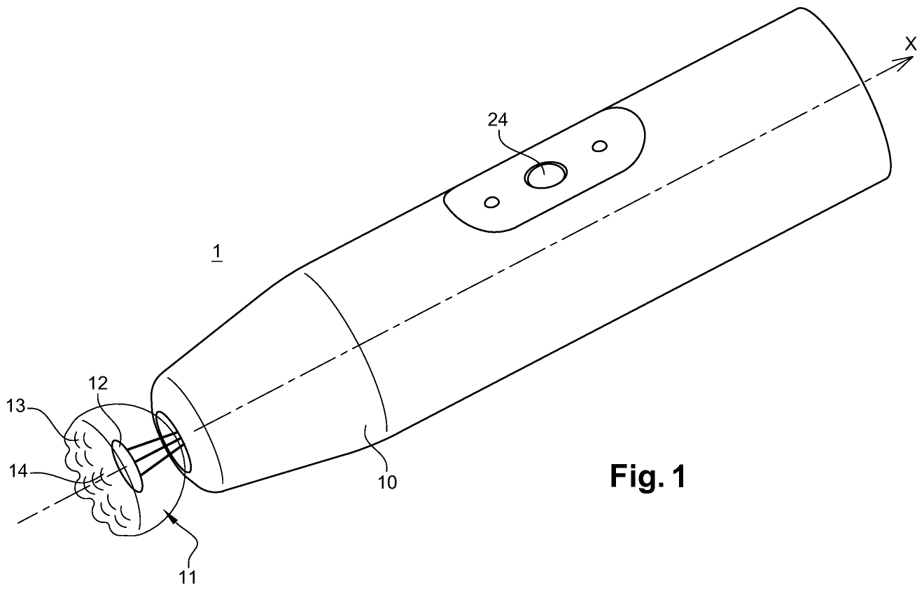

The device 1 shown in FIG. 1 comprises a hand piece 10 and a headpiece 11. It has a longitudinal axis X. The plasma generator is, for example, of DBD ampule type, and in this case, the headpiece 11 may comprise a conductive element 12, which is here a noble ionised gas. The electrode is spaced apart from the dielectrical wall 14 by the noble ionized gas.

The electrode can be supplied with high-voltage pulses generated by a generator 100. On this figure, the projecting elements of the electrode are not represented.

The dielectric wall 14 comprises an external surface provided with reliefs 13.

The skin of the person serves as a counter electrode, and receives a harmless electric current.

The hand piece 10 can be formed as a tube made of plastic or metal, while the hand piece 10 can be removably inserted in its proximal end.

The device can be turned on/off by pressing the on/off button 24.

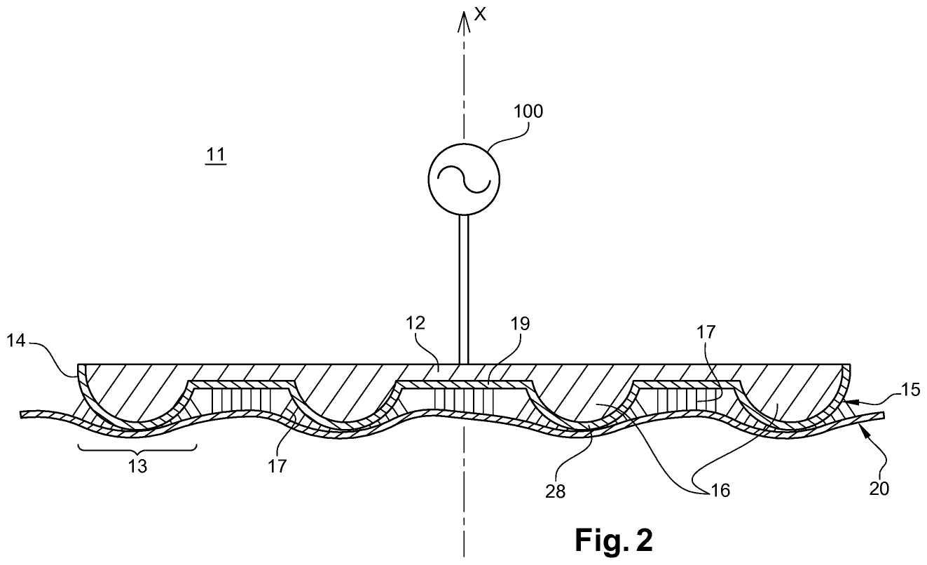

The headpiece 11 shown in FIG. 2 and disposed on the skin 20 of a person, comprises an electrode 12 totally shielded by a dielectric wall 13 that forms an end wall 15 facing the surface to be treated. The electrode 12, which is here the conductive element, is provided with a plurality of projecting conductive elements 16. The projective elements 16 are arranged to fit into a corresponding groove 28 of the dielectric wall 14.

The projecting elements 16 represented are hemisphere, but projective elements such as tooth, spheres, hemispheres, rings, crowns or annular beads, formed on the electrode 12 are other examples of their possible shapes.

The dielectric wall 14 defines an external end wall 15 provided with protruding studs forming reliefs 13 which are integral with the dielectric wall 14. When the reliefs 13 rest on the skin of the person, the gas chambers 17 are delimited between the skin and the surface 19 which is offset with respect to the reliefs 13, and in which the dielectric barrier plasma can form. Its volume might be reduced by means of a pressure applied onto the surface to be treated. Whatever the applied pressure is, there is always a gas chamber in which the plasma is generated.

The dielectric wall 14 can be assembled to the electrode 12 by clamping, sticking, gluing. It can also be sprayed onto the electrode or be injected molded. In other process, the electrode can be printed or sprayed on to the dialectical wall.

In other cases, the ampule can be formed by dielectric material like glass, polymers or else. An electrode and a noble gas can be gapped inside the ampule. The electrical field which is generated can be used to ionized the noble gas during plasma generation.

The invention is not limited to the illustrative embodiments which have just been described, the characteristics of which may be combined with one another as parts of variants which are not illustrated. Any other shape of reliefs and/or dielectric wall could be contemplated.

1. A treatment device for a body surface to be treated using a dielectric barrier plasma, comprising a high-voltage generator connected to a headpiece comprising a conductive element at least partially shielded by a dielectric wall that forms at least one part of an end wall facing the surface to be treated, characterized by the fact that the conductive element is provided with one or more projecting conductive elements and the dielectric wall conforms to the shape of the projecting conductive elements.

2. The treatment device according to claim 1, wherein the conductive element is an electrode.

3. The treatment device according to claim 1, wherein the conductive element is an ionized noble gas.

4. The treatment device according to claim 1, wherein an external surface of the end wall is provided with a plurality of reliefs.

5. The treatment device according to claim 1, wherein the end wall delimits with the body surface to be treated one or more gas chambers when the end wall bears on the surface to be treated, the dielectric barrier plasma being generated in the gas chambers.

6. The treatment device according to claim 1, wherein the thickness of the dielectric element is constant over the whole surface of the end wall facing the surface to be treated.

7. The treatment device according to claim 1, wherein the reliefs comprise a curved external surface.

8. The treatment device according to claim 1, wherein the projecting conductive elements have a concave surface with a concavity turned towards the electrode.

9. The treatment device according to claim 1, wherein the projecting conductive elements have a circular, elliptical, oval or polygonal cross section perpendicular to their longitudinal axis.

10. The treatment device according to claim 1, wherein the plasma is of direct type, in particular DBD type.

11. The treatment device according to claim 1, wherein the plasma is generative violet ray.

12. The treatment device according to claim 1, wherein the reliefs are distributed within the external surface of the end wall.

13. The treatment device according to claim 1, wherein the reliefs are distributed at the periphery of the external surface of the end wall.

14. The treatment device according to claim 1, wherein the reliefs are regularly distributed on the external surface of the end wall.

15. A cosmetic process for treating a body surface using a dielectric barrier plasma, wherein a device according to claim 1 is moved on the body surface.

16. Use of a device according to claim 1 for preventing and/or treating aesthetic disorders of a healthy scalp or of a healthy skin.
